(19) United States
(12) Patent Application Publication
(10) Pub. No.: US 2014/0294922 A1
(43) Pub. Date: Oct. 2, 2014

(54) COMPOUND WITH ANTIBACTERIAL ACTIVITY

(71) Applicant: Aleardo Koverech, Rome (IT)

(72) Inventor: Aleardo Koverech, Rome (IT)

(73) Assignee: SIGMA-TAU INDUSTRIE FARMACEUTICHE RIUNITE S.p.A., Rome (IT)

(21) Appl. No.: 13/996,406

(22) PCT Filed: Dec. 1, 2011
(86) PCT No.: PCT/EP2011/071479

§ 371 Date: Oct. 8, 2013

(30) Foreign Application Priority Data
Dec. 22, 2010 (EP) 10196431.0

(51) Int. Cl. A61K 31/573

(52) U.S. Cl. CPC A61K 31/573
USPC 424/450; 514/180

(57) ABSTRACT

It is described the use of betamethasone as antibacterial agent.

The present invention relates to betamethasone as antibacterial agent. Betamethasone is a moderately potent glucocorticoid steroid with anti-inflammatory and immunosuppressive properties. Unlike other drugs with these effects, betamethasone does not cause water retention. It is applied as a topical cream, ointment, foam, lotion or gel to relieve skin irritation, such as itching and flaking from eczema. Betamethasone sodium phosphate is sometimes prescribed as an intramuscular injection (I.M.) for itching from various ailments including allergic reactions to poison ivy and similar plants. It is available in a number of compound forms: betamethasone dipropionate (branded as Diprosone™, Diprolene™ and others), sodium phosphate and valerate (branded as Betnovate™, Celestone™ and others). Betamethasone dipropionate and salicylic acid can be used as a treatment for local psoriasis. Betamethasone sodium phosphate is used orally and via injection with the same indications as other steroids. It is also used to stimulate fetal lung maturation and to decrease the incidence and mortality from intracranial hemorrhage in premature infants. The use of betamethasone as antibacterial agent it is not known in the medical field.

An antibacterial agent is a substance that kills bacteria or slows their growth, it is sometimes used as a synonym for “antibiotic”, but this term is more properly applied to the broader category of antimicrobial compounds.

Oral antibiotics are simply ingested, while intravenous antibiotics are used in more serious cases, such as deep-seated systemic infections. Antibiotics may also sometimes be administered topically, as with eye drops or ointments. Although antibiotics are generally considered safe and well-tolerated, they have been associated with a wide range of adverse effects. There are various side-effects that can be very serious depending on the antibiotics used and the microbial organisms targeted. The safety profiles of newer medications may not be as well established as those that have been in use for many years. Adverse effects can range from fever and nausea to major allergic reactions including photodermatitis and anaphylaxis.

Other side-effects can result from interaction with other drugs, such as elevated risk of tendon damage from administration of a quinolone antibiotic with a systemic corticosteroid. Certain antibiotics administered by IV (e.g. aminoglycosides, vancomycin) can cause significant permanent hearing loss. Antibiotics like Penicillin and Erythromycin, which used to be one-time miracle cures are now less effective because bacteria have become more resistant. Antibiotics themselves act as a selective pressure that allows the growth of resistant bacteria within a population and inhibits susceptible bacteria.

Therefore, in the medical field it is a still a perceived need to have available new compounds having antibacterial activity not endowed with the drawbacks of the compounds known in the art.

It is now been found that betamethasone is endowed of unexpected antibacterial activity.

It is therefore an object of the present invention betamethasone or a derivative thereof as an antibacterial agent. In which said bacteria are gram-negative or gram-positive bacteria.

It is a further object of the present invention a method of treatment of bacterial infections which comprises administering to a patient in need thereof a suitable amount of betamethasone or a derivative thereof or a salt thereof.

A non limiting example of betamethasone or a derivative thereof, or a salt thereof, is selected from the group comprising betamethasone sodium phosphate, disodium phosphate, dipropionate and valerate.

In the scopes of the present invention other salts of betamethasone, or derivative thereof, which maintain, the same antibacterial activity are also included.

According to the present invention betamethasone can be administered in liquid, semiliquid, solid, powder, spray or liposomal form; for enteral or parenteral administration; in the form of vial, eye drops, capsule, sachets, ointment, foam, suppository, lotion, gel, spray or liposomal; for oral, topical, ophthalmic, rectal, nasal, vein or intramuscular administration.

Any suitable way of administration of betamethasone according to the present invention includes all the methods of administration well known in the art.

Betamethasone according to the present invention can be used in a dose of 0.001-1000 mg/die; a preferred dose is 0.01-100 mg/die; most preferred dose is 0.1-50 mg/die.

For pediatric use betamethasone can be administered in a dose of 0.01-50 mg/Kg of body weight/die; preferred dose is 0.1-5 mg/Kg of body weight/die; most preferred dose is 0.5-2 mg/Kg of body weight/die.

Different doses can be administered according to the physician experience.

The following non-limiting examples further illustrate the invention.

Antibacterial Activity of Betamethasone Disodium Phosphate (Bentelan™)

Bacterial Strains

83 clinical and reference bacterial strains (40 Gram-negative and 43 Gram-positive strains) where used.

The Gram-negative strains included 15 strains chosen in the genus Pseudomonas and 25 strains chosen among Enterobacteriaceae.

The Gram-positive strains included 15 Staphylococcus aureus, 14 Coagulase-negative staphylococci, 1 enterococcus and 13 streptococci strains.

The strains were chosen to cover the main Gram-positive and Gram-negative genera responsible for the most common opportunistic infections.

Every strain was validated using microbiology and biochemical techniques such as:
Macroscopic, morphology analysis of colonies on plates;
Microscopic analysis of freshly prepared colonies to evidence bacterial morphology (cocci, bacilli etc.);
Gram assay microscopy analysis;
Microbiology analysis via selective culture medium (MSA, McConkey, etc.);
Metabolic analysis via biochemical reactions (catalase, oxidase, coagulase test etc.);
Depending on need, ad hoc identification analysis was carried out via defined diagnostic kits (Api™-tests, Vitek™ instrumentation).

Growth Curves

Time dependence of the bacterial growth curves was followed both in the presence and absence (control) of Bentelan™ at a single concentration.

Bentelan™ injectable formulation was at a concentration of 2 mg/ml the chosen single dose concentration is 1 mg/ml which was obtained by diluting Bentelan™ stock solution with the appropriate 2× Brain Heart Infusion (BHI) culture medium (1:2 dilution) so as to feed the bacteria with the standard growth medium concentration. Bacterial inoculum was made from an over-night culture in the same medium, and appropriately diluting it at the moment of inoculum. The growth was followed over 24 hours, collecting at least 6 time points (0, 2, 4, 6, 8 and 24 h). The bacteria was grown by inoculating 10 mL of sterile growth medium planed in 50 mL disposable tubes, at 37° C., generally with vigorous shaking but for streptococci, where it was omitted, and a 5% CO2 atmosphere was also used.

Readout at given times was optical density (OD).

Once isolated, the strains were stored at −80° C. by using a standard freezing protocol (15% glycerol, v/v).

The same block of experiments was performed by using the single excipients contained in the Bentelan™ formulation, to evaluate their role if any.

MIC Experiments

Minimum Inhibitory Concentration (MIC) assays was performed in two set-up: i) varying Bentelan™ concentration to identify the most appropriate dosage on a chosen subset between the strains that exhibited growth inhibition; ii) using at least one antibiotic for each of the following families: inaorolides, cephalosporins and quinolones in the presence/absence of Bentelan™ at two concentrations (High and Low). Depending on the results obtained in the growth curve excipients control experiments, and/or on the positive synergy components identified, several MIC experimental controls were also performed, these included tests on preservatives/additives present in Bentelan™.

Materials & Reagents

Bentelan™ injectable formulation.

Bacterial Strains

The strains collected were either from clinical isolates, or from DSMZ (Deutsche Sammlung von Mikroorganismen und Zellkulturen) or from ATCC (American Type Culture Collection).

Everyone of the 83 strains was subjected to the validation experiments mentioned above.

### EXAMPLE 1

Antibacterial Activity of Bentelan™ (Betametasone Disodium Phosphate) on Staphylococcus aureus 15 Strains

The ensemble comprised 3 reference (ATCC and DMSZ) and 12 clinical strains; they were classified as MRSA (meticillino resistant Staphylococcus aureus) and MSSA (methicillin-sensitive Staphylococcus aureus).

Characterization verified:

a) bacterial microscopic morphology: grape-like clusters

b) Gram-positive

c) Microbiology analysis via selective medium: Mannitol Salt Agar (MSA)

d) Metabolic biochemical reactivity: Coagulase and catalase positives.

The results obtained, reported in Table 1.

TABLE 1
Antibacterical activity of Bentelan ™ (Betametasone disodium
phosphate) on Staphylococcus aureus, 15 strains
Staphylococcus aureus (15 strains)

| | | OD 600 nm Time (Hours) | | | | | |
|---|---|---|---|---|---|---|---|
| | | 0 | 2 | 4 | 6 | 8 | 24 |
| Strains | Min. | 0.075 | 0.80 | 4.0 | 4.10 | 4.10 | 4.10 |
| S.a. 1-15 | Value | | | | | | |
| not treated | Max. | 0.10 | 1.20 | 7.50 | 7.60 | 7.60 | 7.60 |
| with | Value | | | | | | |
| Bentelan ™ | | | | | | | |
| Strains | Min. | 0.06 | 0.075 | 0.075 | 0.10 | 0.10 | 0.12 |
| S.a. 1-15 | Value | | | | | | |
| treated with | Max. | 0.08 | 0.11 | 1.20 | 1.50 | 0.60 | 0.70 |
| Bentelan ™ | Value | | | | | | |

The results reported above show that Bentelan™ has a clear and evident inhibition effect on the growth of all the strains tested.

### EXAMPLE 2

Antibacterial Activity of Bentelan™ on Coagulase Negative staphylococci (CoNS), 14 Strains.

The ensemble comprises 4 reference (ATCC and DMSZ) and 10 clinical strains.

Characterization verified:

a) bacterial microscopic morphology: grape-like clusters

b) Gram-positive

c) Microbiology analysis via selective medium: Mannitol Salt Agar (MSA)

d) Metabolic biochemical reactivity: coagulase negative and catalase positives.

Among the coagulase negatives strains both Staphylococcus epidermis and Staphylococcus haemolyticus were identified.

The results obtained, reported in Table 2.

TABLE 2
Antibacterical activity of Bentelan ™ on Coagulase negative
Staphylococci (CoNS), 14 strains.
(CoNS) Coagulase negative staphylococci (14 strains)

| | | OD 600 nm Time (Hours) | | | | | |
|---|---|---|---|---|---|---|---|
| | | 0 | 2 | 4 | 6 | 8 | 24 |
| Strains | Min. | 0.08 | 0.08 | 0.09 | 0.20 | 4.10 | 2.50 |
| CoNS 1-14 | Max. | 0.11 | 0.90 | 7.50 | 7.30 | 7.50 | 8.0 |
| not treated with | | | | | | | |
| Bentelan ™ | | | | | | | |
| Strains | Min. | 0.06 | 0.07 | 0.04 | 0.04 | 0.07 | 0.012 |
| CoNS 1-14 | Max. | 0.09 | 0.20 | 0.30 | 0.50 | 0.60 | 0.70 |
| treated with | | | | | | | |
| Bentelan ™ | | | | | | | |

The results reported above show that Bentelan™ has a clear and evident inhibition effect on the growth of all the strains tested.

### EXAMPLE 3

Antibacterial Activity of Bentelan™ on Streptococcus spp. 13 Strains

The ensemble comprises 2 reference (ATCC and DMSZ) and 11 clinical strains.

Characterization verified:

a) bacterial microscopic morphology: chain

b) Gram-positive

c) Microbiology analysis via selective medium: blood agar

d) Metabolic biochemical reactivity: catalase negative.

Among the coagulase negative strains analysed both Streptococcus viridans and Streptococcus β-emolytic were identified.

In Table 3 is shown the growth curve for 13 strains in the presence and absence of Bentelan™.

TABLE 3
Antibacterical activity of Bentelan ™ on Streptococcus spp. 13 strains
Streptococcus spp. (13 strains)

| | | OD 600 nm Time (Hours) | | | | | |
|---|---|---|---|---|---|---|---|
| | | 0 | 2 | 4 | 6 | 8 | 24 |
| Strains | Min. | 0.01 | 0.02 | 0.05 | 0.075 | 0.25 | 0.50 |
| Strepto 1-13 | Max. | 0.075 | 0.12 | 0.90 | 2.30 | 2.40 | 2.40 |
| not treated with | | | | | | | |
| Bentelan ™ | | | | | | | |
| Strains | Min. | 0.01 | 0.01 | 0.01 | 0.01 | 0.01 | 0.01 |
| Stepto 1-13 | Max. | 0.08 | 0.07 | 0.07 | 0.07 | 0.08 | 0.07 |
| treated with | | | | | | | |
| Bentelan ™ | | | | | | | |

The results reported above show that Bentelan™ has a clear and evident inhibition effect on the growth of all the strains tested.

### EXAMPLE 4

Antibacterial Activity of Bentelan™ on 1 Strain of Enterococcus spp.

Characterization verified:

a) bacterial microscopic morphology: cocci

b) Gram-positive

c) Microbiology analysis via selective medium: Enterococcusel™ Agar.

In Table 4 is shown the growth curve for on 1 strain of Enterococcus in the presence and absence of Bentelan™.

TABLE 4
Antibacterical activity of Bentelan ™ on 1 strain of Enterococcus spp.
Enterococcus spp. (1 strain)

| | OD 600 nm Time (Hours) | | | | | |
|---|---|---|---|---|---|---|
| | 0 | 2 | 4 | 6 | 8 | 24 |
| Enterococcus | 0.90 | 1.10 | 6.00 | 6.50 | 7.50 | 7.40 |
| spp. strain not | | | | | | |
| treated with | | | | | | |
| Bentelan ™ | | | | | | |
| Enterococcus | 0.075 | 0.10 | 0.20 | 0.16 | 0.15 | 0.25 |
| spp. strain | | | | | | |
| treated with | | | | | | |
| Bentelan ™ | | | | | | |

Bentelan™ shows an inhibition effect on the growth of the line tested.

### EXAMPLE 5

Antibacterial Activity of Bentelan™ on Pseudomonas spp., 15 Strains

The ensemble comprises 2 reference strains (ATCC and DMSZ) and 13 clinical strains. Among the strains several Pseudomonas aeruginosa were present.

Characterization verified:

a) bacterial microscopic morphology: bacilli

b) Gram-negative

c) Microbiology analysis via solid medium: tryptic soy agar (TSA) and Mc-Conkey agar d) Metabolic biochemical reactivity; oxidase positive.

The results obtained are reported in Table 5.

TABLE 5
Antibacterical activity of Bentelan ™ on Pseudomonas spp., 15
strains.
Pseudomonas spp. (15 strains)

| | | OD 600 nm Time (Hours) | | | | | |
|---|---|---|---|---|---|---|---|
| | | 0 | 2 | 4 | 6 | 8 | 24 |
| Strains | Min. | 0.035 | 0.25 | 0.50 | 0.80 | 1.0 | 2.0 |
| Pseudo. 1-15 | Max. | 0.075 | 0.75 | 1.50 | 2.0 | 2.10 | 4.50 |
| not treated | | | | | | | |
| with entelan ™ | | | | | | | |
| Strains | Min. | 0.01 | 0.01 | 0.01 | 0.01 | 0.01 | 0.01 |
| Pseudo. 1-15 | Max. | 0.025 | 0.024 | 0.024 | 0.024 | 0.025 | 0.025 |
| treated with | | | | | | | |
| Bentelan ™ | | | | | | | |

Bentelan™ shows an inhibition effect on the growth of the line tested.

### EXAMPLE 6

Antibacterial Activity of Bentelan™ on Bacterial Strains Belonging to the Family of Enterobacteriaceae, 25 strains

The ensemble comprises 2 reference (ATCC and DMSZ) and 23 clinical strains. Among the clinical strains several Escherichia coli and others belonging to Klebsiella spp. were present. Both species were lactose-fermenting.

Characterization verified:

a) bacterial microscopic morphology: bacilli

b) Gram-negative

c) Microbiology analysis via selective sold medium: Mc-Conkey agar.

d) Metabolic biochemical reactivity: oxidase negative.

The results obtained are reported in Table 6.

TABLE 6
Antibacterical activity of Bentelan ™ on bacterial strains belonging
to the family of Enterobacteriaceae, 25 strains
Enterobacteriaceae (25 strains)

| | | OD 600 nm Time (Hours) | | | | | |
|---|---|---|---|---|---|---|---|
| | | 0 | 2 | 4 | 6 | 8 | 24 |
| Strains | Min. | 0.035 | 0.75 | 2.5 | 2.4 | 2.5 | 3.5 |
| Enterobact. 1-25 | Max. | 0.50 | 2.0 | 5.0 | 4.0 | 5.0 | 8.0 |
| not treated with | | | | | | | |
| Bentelan ™ | | | | | | | |
| Strains | Min. | 0.01 | 0.01 | 0.01 | 0.01 | 0.01 | 0.01 |
| Enterobact. 1-25 | Max. | 0.06 | 0.07 | 0.075 | 0.08 | 0.085 | 0.075 |
| treated with | | | | | | | |
| Bentelan ™ | | | | | | | |

Bentelan™ shows an inhibition effect on the growth of the line tested.

Betamethasone is a compound well known in the medical field branded as Bentelan™; Diprosone™, Diprolene™; Betnovate™ or Celestone™.

1-10. (canceled)

11. A method of treating bacterial infections, comprising administering to a patient in need thereof a suitable amount of betamethasone or a derivative thereof or a salt thereof.

12. The method of claim 11, wherein for the bacterial infection is a gram-negative or gram-positive bacterial infection.

13. The method of claim 11, wherein the derivative or salt of betamethasone is selected from the group consisting of betamethasone sodium phosphate, disodium phosphate, dipropionate and valerate.

14. The method of claim 11, wherein the betamethasone is in liquid, semiliquid, solid, powder, spray or liposomal form, for enteral or parenteral administration.

15. The method of claim 11, wherein the betamethasone is in the form of eye drops, capsule, sachets, ointment, foam, suppositories, lotion, gel, or spray.

16. The method of claim 11, wherein the betamethasone is administered via a route selected from the group consisting of oral, topical, ophthalmic, rectal, nasal, ocular, vein and intramuscular administration.

17. The method of claim 11, wherein the betamethasone is administered at a dose of 0.001-1000 mg/day.

18. The method of claim 17, wherein the betamethasone is administered at a dose of 0.01-100 mg/day.

19. The method of claim 18, wherein the betamethasone is administered at a dose of 0.10-50 mg/day.

20. The method of claim 11, wherein the betamethasone is administered at a dose of 0.01-50 mg/Kg of body weight/day.

21. The method of claim 11, wherein the betamethasone is administered at a dose of 0.10-5 mg/Kg of body weight/day.

22. The method of claim 11, wherein the betamethasone is administered at a dose of 0.5-2 mg/Kg of body weight/day.
